(19) United States
(12) Patent Application Publication
(10) Pub. No.: US 2026/0289948 A1
(43) Pub. Date: Sep. 24, 2026

(54) INFERENCE OF CARTILAGE SEGMENTATION IN COMPUTED TOMOGRAPHY SCANS

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Michel Goncalves ALMEIDA ANTUNES, Coimbra (PT); Miguel MARQUES, Montemor-o-Velho (PT); João Pedro DE ALMEIDA BARRETO, Coimbra (PT); Carolina DOS SANTOS RAPOSO, Coimbra (PT)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 19/666,467

(22) Filed: May 4, 2026

Related U.S. Application Data
(63) Continuation of application No. PCT/US2024/061014, filed on Dec. 19, 2024.
(60) Provisional application No. 63/612,470, filed on Dec. 20, 2023.

(51) Int. Cl. G06V 10/26; G06T 7/174; G06T 7/30; G06V 10/774; G06V 10/82; G06V 20/70

(52) U.S. Cl. CPC G06V 10/26; G06T 7/174; G06T 7/30; G06V 10/774; G06V 10/82; G06V 20/70; G06T 2207/10081; G06T 2207/10088; G06T 2207/20081; G06T 2207/20084; G06T 2207/30008; G06V 2201/033

(57) ABSTRACT

Some examples are directed to a processor-implemented methods of training a neural network for soft-tissue labeling of computed tomography (CT) images, neural networks trained according to one or more of the processor-implemented methods, processor-implemented methods of soft-tissue-labeling a CT image, and systems for labeling soft-tissue in computed tomography images.

### CROSS-REFERENCE TO RELATED APPLICATIONS

This application is a continuation of and claims the benefit of International Application No. PCT/US2024/061014 filed on Dec. 19, 2024 and titled “INFERENCE OF CARTILAGE SEMENTATION IN COMPUTED TOMOGRAPHY SCANS”, which claims the benefit of U.S. Provisional Patent Application Ser. No. 63/612,470 filed on Dec. 20, 2023 and titled “INFERENCE OF CARTILAGE SEGMENTATION IN COMPUTED TOMOGRAPHY SCANS.” The contents of these applications are incorporated herein by reference as if reproduced in full herein.

### BACKGROUND

Medical imaging of anatomical structures using computed tomography (CT) or magnetic resonance imaging (MRI) technologies can provide detailed information about the anatomical structures, useful for diagnosis, modeling, and research.

Segmentation of a medical image refers to a process by which different anatomical structures captured in a given medical image, such as different soft-tissue structures and different hard-tissue structures, are distinguished from one another and from background. Segmentation may be done manually, by experienced radiologists or other personnel while viewing the medical image, or may be done at least partially automatically, by computer systems configured to segment the medical images. Segmentation of a medical image is generally intended to result in each pixel of the medical image being labeled as a part of one anatomical structure or of another, or as background.

For example, in a medical image of a knee joint encoded as pixels, after a successful segmentation, several pixels may be labeled as being a part of a tibia bone, several other pixels may be labeled as being a part of tibial cartilage, several other pixels may be labeled as being a part of a femur bone, several other pixels may be labeled as being a part of femoral cartilage, and several other pixels may be labeled simply as background. By differentially labeling pixels according to the anatomical structure of which they are a part, the anatomical structures themselves can be distinguished from and sharply delineated in the medical images from one another. Accurate delineation of anatomical structures in individual image slices of a set of medical images is useful for injury or pathology diagnoses, and is also useful when generating accurate three-dimensional models of the anatomical structures and their interrelationships, for aiding diagnoses of injury, for planning for repair, and for surgical guidance during repair.

Some medical imaging technologies, or “modalities”, are better suited than others for capturing details of certain kinds of anatomical structures. For example, MRI technology can be excellent for capturing details of soft-tissue anatomical structures such as cartilage as well as those of hard-tissue anatomical structures such as bone. However, CT technology, while excellent for capturing details of hard-tissue anatomical structures, tends to far less effective for capturing details of soft-tissue anatomical structures. Because CT technology is not as effective for capturing details of soft-tissue anatomical structures, fulsome segmentation of CT images to accurately label the soft-tissue anatomical structures can be difficult or impractical to do, either manually or automatically.

### SUMMARY

One example is a processor-implemented method of training a neural network for soft-tissue labeling comprising: creating a training set of soft-tissue labeled computed tomography (CT) images comprising: collecting, from at least one database, pairs of image sets for each of a plurality of individuals, each pair comprising both a CT image set of an individual's anatomy and a magnetic resonance (MR) image set of the individual's anatomy; and for each of the pairs: creating a first 3D model of both bone and soft-tissue of the individual's anatomy using the MR image set; creating a second 3D model of bone of the individual's anatomy using the CT image set; generating a 3D transformation between the bone in the first 3D model and the bone in the second 3D model; labeling soft-tissue in at least one CT image in the CT image set based on an application of the 3D transformation to labeled soft-tissue in the first 3D model, thereby to create at least one soft-tissue-labeled CT image; and augmenting the training set with the at least one soft-tissue-labeled CT image; and training the neural network using the training set to label soft-tissue in CT images.

In the example processor-implemented method, labeling soft-tissue in at least one CT image in the CT image set based on an application of the 3D transformation to labeled soft-tissue in the first 3D model may comprise: applying the 3D transformation to the soft-tissue in the first 3D model to label contents of the second 3D model as soft-tissue thereby to create an augmented second 3D model; and creating the at least one soft-tissue-labeled CT image using the augmented second 3D model.

In the example processor-implemented method, generating a 3D transformation between the bone in the first 3D model and the bone in the second 3D model may comprise: applying a global registration process to determine an initial alignment transformation between the bone in the first 3D model and the bone in the second 3D model; and applying an iterative closest point (ICP) process to the initial alignment transformation thereby to generate the 3D transformation.

In the example processor-implemented method, applying a global registration process to determine an initial alignment transformation between the bone in the first 3D model and the bone in the second 3D model may comprise applying a 2PNS (2-Point-Normal Sets) process to determine the initial alignment transformation between the bone in the first 3D model and the bone in the second 3D model.

In the example processor-implemented method, the neural network may be a U-net neural network.

In the example processor-implemented method, creating a first 3D model of both bone and soft-tissue of the individual's anatomy using the MR image set may comprise: receiving at least bone labeling and soft-tissue labeling corresponding to MR images in the MR image set; and applying a Marching Cubes process to the bone and soft-tissue labeling of the MR images.

In the example processor-implemented method, creating a second 3D model of bone of the individual's anatomy using the CT image set may comprise: receiving at least bone labeling corresponding to CT images in the CT image set; and applying a Marching Cubes process to the bone labeling of the CT images.

In the example processor-implemented method, the individual's anatomy may be a knee joint, and labeling soft-tissue in at least one CT image in the CT image set may comprise labeling femoral cartilage and tibial cartilage of the knee joint.

Another example is a non-transitory processor-readable medium embodying processor-readable program code executable by at least one processor to carry out one or more of the processor-implemented methods set forth above.

Another example is a neural network trained according to one or more of the processor-implemented methods set forth above.

In the example, the neural network may be a U-net neural network.

Another example is a processor-implemented method of soft-tissue-labeling a computed tomography (CT) image. The method may comprise: providing an input CT image as input to the neural network set forth above; and receiving, from the neural network, a soft-tissue-labeling of the input CT image.

Another example is a system for labeling soft-tissue in a computed tomography (CT) image. The system may comprise: at least one processor; and a memory coupled to the at least one processor, the memory storing instructions that, when executed by the at least one processor, cause the at least one processor to: provide an input CT image as input to the neural network set forth above; and receive, from the neural network, a soft-tissue-labeling of the input CT image.

In the one or more processor-implemented methods set forth above, the soft-tissue may be cartilage.

Another example is a processor-implemented method of training a neural network for soft-tissue labeling. The processor-implemented method may comprise: creating a training set of soft-tissue-labeled computed tomography (CT) images comprising: collecting, from at least one database, pairs of images for each of a plurality of individuals, each pair comprising both a CT image of an individual's anatomy and a magnetic resonance (MR) image of the individual's anatomy; and for each of the pairs: generating a 2D transformation between bone in the MR image and bone in the CT image; labeling soft-tissue in the CT image based on an application of the 2D transformation to labeled soft-tissue in the MR image, thereby to create a soft-tissue-labeled CT image; and augmenting the training set with the soft-tissue-labeled CT image; and training the neural network using the training set to label soft-tissue in CT images.

In the processor-implemented method, labeling soft-tissue in the CT image based on an application of the 2D transformation to labeled soft-tissue in the MR image may comprise: applying the 2D transformation to the soft-tissue in the MR image to label contents of the CT image as soft-tissue thereby to create the soft-tissue-labeled CT image.

In the processor-implemented method, generating a 2D transformation between the bone in the MR image and the bone in the CT image may comprises: applying a global registration process to determine an initial alignment transformation between the bone in the MR image and the bone in the CT image; and applying an iterative closest point (ICP) process to the initial alignment transformation thereby to generate the 2D transformation.

In the processor-implemented method, the neural network may be a U-net neural network.

In the processor-implemented method, the individual's anatomy may be a knee joint, wherein labeling soft-tissue in the CT image may comprise labeling femoral cartilage and tibial cartilage of the knee joint.

Another example is a non-transitory processor-readable medium embodying processor-readable program code executable by at least one processor to carry out one or more of the processor-implemented methods set forth above.

Another example is a neural network trained according to one or more of the processor-implemented methods set forth above.

In an example, the neural network may be a U-net neural network.

Another example is a processor-implemented method of soft-tissue-labeling a computed tomography (CT) image. The method may comprise: providing an input CT image as input to the neural network; and receiving, from the neural network, a soft-tissue-labeling of the input CT image.

Another example is a system for labeling soft-tissue in a computed tomography (CT) image. The system may comprise: at least one processor; and a memory coupled to the at least one processor, the memory storing instructions that, when executed by the at least one processor, cause the at least one processor to: provide an input CT image as input to the neural network; and receive, from the neural network, a soft-tissue-labeling of the input CT image.

In one or more of the processor-implemented methods set forth above, the soft-tissue may be cartilage.

Another example is a processor-implemented method of soft-tissue labeling an input CT image of an individual's anatomy, comprising: generating, by a neural network based on the input CT image, a respective output synthetic magnetic resonance (MR) image of the individual's anatomy, wherein the neural network is trained using a generative adversarial approach, using at least CT training images and MR training images, to receive CT images of individuals' anatomy and generate respective output synthetic MR images of the individuals' anatomy; receiving at least soft-tissue labeling corresponding to the respective output synthetic MR image; and applying at least the soft-tissue labeling from the respective output synthetic MR image to the input CT image thereby to soft-tissue label the input CT image.

In an example, receiving at least soft-tissue labeling corresponding to the output synthetic MR image may comprise: providing the output synthetic MR image to an automatic image segmentation system; and receiving at least the soft-tissue labeling corresponding to the output synthetic MR image from the automatic image segmentation system.

In an example, applying at least the soft-tissue labeling from the output synthetic MR image to the input CT image thereby to soft-tissue label the input CT image may comprise: resampling a segmentation volume of the output synthetic MR image using the input CT image as a reference.

The processor-implemented method may further comprise: configuring the neural network as a first generative adversarial network (GAN) interconnected with a second GAN, wherein the first GAN is trained to generate a synthetic output MR image from a first GAN input, and the second GAN is trained to generate a synthetic CT image from a second GAN input; and training the neural network by, for each of a plurality of cycles: providing, as a first GAN input, a synthetic CT image generated by the second GAN thereby to generate, as a first GAN output, a corresponding synthetic MR image; providing, as a second GAN input, a synthetic MR image generated by the first GAN thereby to generate, as a second GAN output, a corresponding synthetic CT image; determining a first measure of difference between the second GAN output and the first GAN input; determining a second measure of difference between the first GAN output and the second GAN input; and modifying the first GAN and the second GAN based at least on the first measure of difference and the second measure of difference, wherein the modifying is conducted to reduce, over successive cycles, magnitudes of the first measure of difference and the second measure of difference.

In an example, the processor-implemented method may further comprise: during training of the neural network: receiving bone labeling for the synthetic CT image generated by the second GAN; and using the bone labeling as regularization during the modifying.

In an example, the neural network is a U-net neural network.

In an example, the individual's anatomy may be a knee joint and the soft-tissue-labeling includes labeling for femoral cartilage and labeling for tibial cartilage.

Another example is a non-transitory processor-readable medium embodying processor-readable program code executable by at least one processor to carry out one or more of the processor-implemented methods set forth above.

Another example is a neural network trained according to one or more of the processor-implemented methods set forth above.

In an example, the neural network is a U-net neural network.

Another example is a processor-implemented method of soft-tissue-labeling a computed tomography (CT) image. The method may comprise: providing an input CT image as input to the neural network; and receiving, from the neural network, a soft-tissue-labeling of the input CT image.

Another example is a system for labeling soft-tissue in a computed tomography (CT) image, the system comprising: at least one processor; and a memory coupled to the at least one processor, the memory storing instructions that, when executed by the at least one processor, cause the at least one processor to: provide an input CT image as input to the neural network; and receive, from the neural network, a soft-tissue-labeling of the input CT image.

In an example, the soft-tissue may be cartilage.

### BRIEF DESCRIPTION OF THE DRAWINGS

For a detailed description of example embodiments, reference will now be made to the accompanying drawings in which:

FIG. 1A shows an example MR image of a knee joint of an individual human patient;

FIG. 1B shows is an example CT image of the same knee joint of FIG. 1A;

FIG. 2A shows an example unsegmented MR image of the knee joint of an individual human patient;

FIG. 2B shows the MR image of FIG. 2A, after segmentation;

FIG. 3A shows an example CT image of a knee joint of an individual human patient without contrast processing;

FIG. 3B shows the example CT image of FIG. 3A after contrast processing using a local windowing technique;

FIGS. 4A, 4B, and 4C show an overall example pipeline that uses a neural network capable of generating a synthetic MR image from an input CT image;

FIG. 5 shows various CT and CT enlarged/zoomed-in images illustrative of comparison of an input CT image with a pseudo ground-truth segmentation and each of three different automatic segmentations determined according to examples disclosed herein;

FIG. 6 shows a computer-implemented method of training a neural network for soft-tissue labeling, in accordance with at least some examples;

FIG. 7 shows steps of the method that may be conducted during creating the training set of soft-tissue labeled CT images in the method of FIG. 6, according to an example;

FIG. 8 shows steps of the method that may be conducted during creating the training set of soft-tissue labeled CT images in the method of FIG. 6, according to an alternative example;

FIG. 9 shows a computer-implemented method of soft-tissue labeling an input CT image of an individual's anatomy, in accordance with an example; and

FIG. 10 shows an example computer system that may be configured to execute the processor-implemented methods disclosed herein, and that may be configured to implement the systems disclosed herein.

### DEFINITIONS

Various terms are used to refer to particular system components. Different companies may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection or through an indirect connection via other devices and connections.

Herein, examples are provided in which medical images of human knee joints, each having a tibia bone, a femur bone, a patella bone, as well as tibial cartilage and femoral cartilage, are described and depicted. It will be appreciated that the images of human knee joints are provided only as useful examples, and that the principles described and depicted in the present application would be understood as pertaining more widely to images of other anatomical structures, such as musculoskeletal anatomical structures, for which segmentation of soft-tissue and hard-tissue in medical images may be desirable.

In the present description, the meaning of soft-tissue may be regarded as it is generally used by the person of ordinary skill in the art, to encompass kinds of anatomical structures that are generally distinguished from anatomical structures such as bones and teeth in that soft-tissue is not hardened by ossification or calcification. Examples of different kinds of soft-tissue may include cartilage, muscle, tendons, ligaments, fat, fibrous tissue, lymph and blood vessels, fasciae, and synovial membranes. It will be appreciated that an imaging modality may be capable of representing different kinds of soft-tissue differently due to differential interactions of such different kinds of soft-tissue with the imaging modality equipment and the manner in which it is configured. For example, an imaging modality configured in a particular way may generate images that represent/depict cartilage of a joint in a different manner than muscle of the same joint. Therefore, in the present description, references to labeling of soft-tissue during the process of segmentation of a medical image of an anatomical structure is intended to indicate the labeling of a particular kind of soft-tissue as distinct from hard-tissue, from a particular kind of hard-tissue and/or from another kind of soft-tissue. For example, labeling of soft-tissue in a medical image may refer to labeling of individual pixels as cartilage, or even more specifically as femoral cartilage or tibial cartilage, with other pixels in the medical image being labelled as bone, or even more specifically as femoral bone or tibial bone, with still other pixels in the medical image being labelled as background. The other pixels labelled as background in such an example may, in reality for example, coincide with muscle but are to be regarded, for the purpose of image processing and labeling for a particular application, as background pixels so that just the cartilage and bone can be further processed and/or studied. Alternatives are possible.

In the present description, examples are provided in which the soft-tissue to be labeled is cartilage and the hard-tissue to be labeled is bone. It will be appreciated that applications of the methods and systems described and depicted herein may arise in which the soft-tissue to be labeled is not cartilage, such as for example in which the soft-tissue is instead muscle, and the hard-tissue is bone, or is some other hard-tissue such as teeth. It will be appreciated that applications of the methods and systems described and depicted herein may arise in which the soft-tissue to be labelled is cartilage and muscle, and the cartilage is to be labeled differently from the muscle so as to enable the differently-labeled muscle and cartilage to be differentially processed and/or studied. Variations are possible.

### DETAILED DESCRIPTION

The following discussion is directed to various examples. Although one or more of these examples may be preferred, the examples disclosed should not be interpreted, or otherwise used, as limiting the scope of the disclosure, including the claims. In addition, one skilled in the art will understand that the following description has broad application, and the discussion of any example is meant only to be exemplary of that example, and not intended to intimate that the scope of the disclosure, including the claims, is limited to that example.

Computed tomography (CT) is a medical imaging technique that uses X-ray technology to create detailed cross-sectional images of a body. CT imaging is commonly used in various musculoskeletal (MSK) specialties for diagnostic and surgical planning purposes. CT scans generate CT images that facilitate accurate visualization and diagnosis of bone-related conditions because of their excellent ability to depict bone structures with high precision and clarity. However, CT scans have several limitations concerning the visualization and diagnosis of soft-tissue. Soft-tissue, such as cartilage, is not depicted as distinctly in CT images as is bone. This may present challenges to perceiving and evaluating such soft-tissue accurately. Specially, using CT alone, cartilage is difficult to visualize and diagnose.

In situations where cartilage evaluation is required, Magnetic Resonance Imaging (MRI), a technology for cross-sectional imaging of a body that uses magnetic field, field gradients, and radiofrequency technology to conduct MRI scans, is usually the standard in clinical practice. MRI scans generate MR images that facilitate accurate visualization and diagnosis of soft-tissue conditions, such as cartilage conditions, because of their excellent ability to depict both bone structures and soft-tissue structures with high precision and clarity. In standard-of-care (SoC), the assessment of the cartilage condition of the knee is important for diagnosing osteoarthritis (OA), which occurs when the cartilage between bones wears or breaks due to injury or disease.

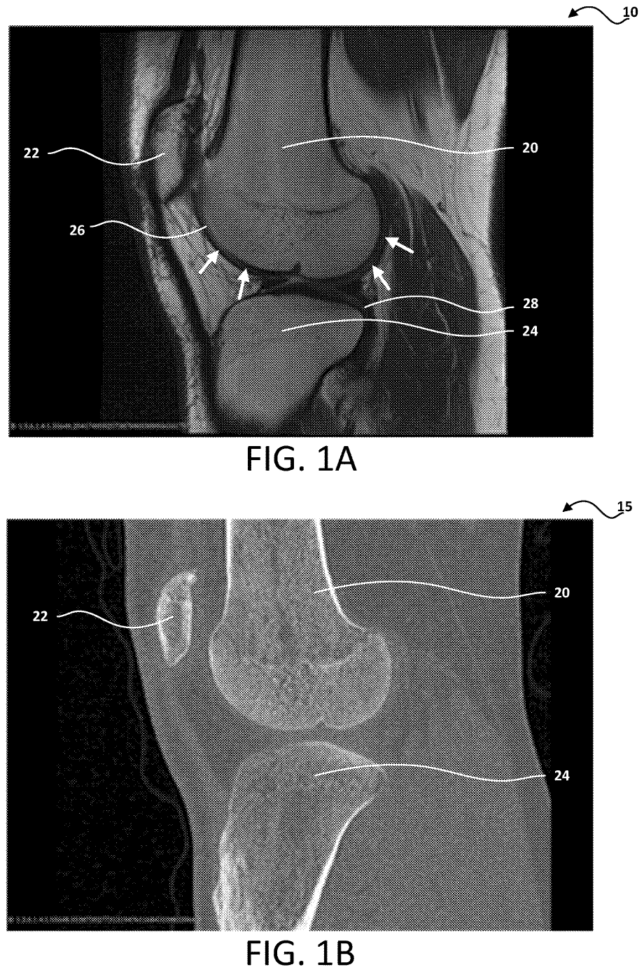

FIG. 1A is an example MR image 10 of a knee joint of an individual human patient, including portions of a femur bone 20, patella bone 22, tibia bone 24, femoral cartilage 26, tibial cartilage 28, and other features. White arrows overlie the MR image and point to some of the femoral cartilage 26, located as a dark region surrounding a distal region of the femur bone 20. The femoral cartilage 26 pointed to by the white arrows is clearly visible and sharply-defined in the MR image 10, enabling clear visual contrast with the femur bone 20 and with other anatomical features of the knee joint. FIG. 1B is an example CT image 15 of the same knee joint of the same patient. While the femur bone 20, patella bone 22, and tibia bone 24 are clearly visible and sharply-defined in the CT image 15, the soft-tissue portions, such as the femoral cartilage, is very difficult to visually discern or distinguish, and thus to unambiguously identify or locate.

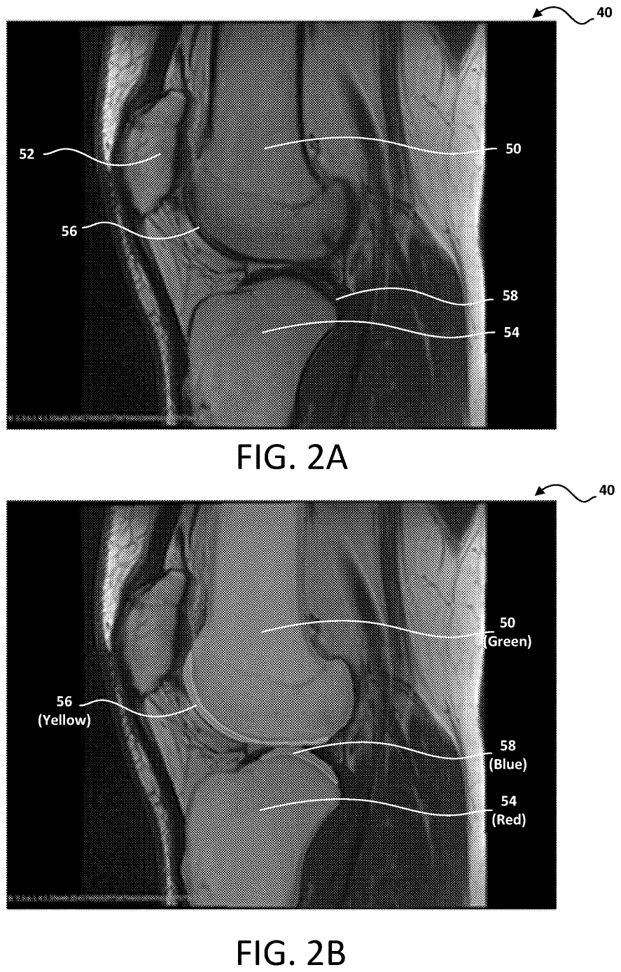

Automatic segmentation in CT and MRI involves the delineation and extraction of anatomical structures and tissues captured in CT images or MR images, allowing for a better understanding of the musculoskeletal system. FIG. 2A is an example unsegmented—and thus unlabeled-MR image 40 of the knee joint of a patient, including portions of a femur bone 50, patella bone 52, tibia bone 54, femoral cartilage 56, tibial cartilage 58, and other features. FIG. 2B is the MR image 40 after segmentation, and thus associated with labeling of its pixels as part of an anatomical structure (or background). In particular, in FIG. 2B, segmentation labels corresponding to each pixel have been used to present the pixels of different anatomical structures in different colors. For example, the pixels labeled as femur bone 50 are presented in the color green, the pixels labeled as femoral cartilage 56 are presented in the color yellow, the pixels labeled as tibia bone 54 are presented in the color red, and the pixels labeled as tibial cartilage 58 are presented in the color blue. Assigning to at least these pixels a label corresponding to an anatomical structure aids in diagnosis, assists in treatment planning, supports image-guided interventions, and drives research. This can enhance clinical decision-making, improve patient outcomes, and contribute to advancements in MSK specialties.

At the time of this writing, the state-of-the-art processes for automatic segmentation of medical images—that is, image segmentation not solely manually conducted by a radiologist or other personnel but instead done with the aid of at least one computer processor conducting image processing of the medical images-make use of deep learning (DL) technology. With DL technology, it is typical for a neural network to trained, using large amounts of labeled data as training data, to be competent at automatic segmentation. In medical image segmentation such training data typically consists in medical images that have been manually annotated (i.e., “labeled”) by health-care professionals or individuals having appropriate medical training.

However, manual annotation of medical images, even for use in providing training data for training a DL system to be competent at automatic segmentation, is generally a time-consuming and error-prone process. Furthermore, manual annotators, even those having extensive experience, generally struggle to very accurately segment soft-tissue such as cartilage in CT images because, as discussed previously in connection with FIG. 1A, perceiving the boundaries of such cartilage from CT images may be very difficult or may be practically unfeasible. As such, it is generally understood by those familiar with annotation that manual annotation of soft-tissue in CT images can, in general, be done only in an approximate manner such that boundaries of such soft-tissue are not fully delineated to a high degree of confidence. In turn, training a DL system using only such approximately-labeled CT training images may result in the DL system not developing sufficient competence for accurate and useful automatic segmentation.

Various approaches to improving the efficiency and accuracy of training of DL systems have been attempted.

Such approaches include cross-modality synthesis, in which CT is synthesized from MRI or MRI is synthesized from CT. As described herein, CT and MRI are two widely used imaging modalities in medical diagnostics. Both modalities provide valuable information about the internal structures of the body. However, they differ in several aspects, including their imaging principles and associated risks.

For example, CT imaging involves the use of X-rays, which are a form of ionizing radiation. X-rays are directed through the anatomy, and detectors measure the amount of radiation that passes through different tissues. This information is processed by a computer to generate cross-sectional images of the anatomy. The ionizing radiation used during CT scans carries potential risks, particularly with repeated or excessive exposure, as the radiation can damage DNA and increase the risk of developing cancer.

On the other hand, MRI utilizes a different imaging mechanism that does not involve ionizing radiation, and instead relies on a magnetic field and radiofrequency waves to create detailed images of the body's structures. MRI is considered a safe imaging modality. However, despite the advantages of MRI over CT in terms of safety, there are instances where CT imaging is still necessary due to its unique capabilities in visualizing certain anatomical details and pathologies. Specifically, bone boundaries, injuries and deformations are very clear in CT images. However, due to its ionizing radiations, researchers have been exploring techniques to compute CT-like images from MRI data. Such techniques aim to bridge the gap between the two modalities and provide an alternative to CT scanning that is not associated with the risks of ionizing radiation. The use of Generative Adversarial Networks (GANs) for translating MRIs to CTs have been proposed.

Generating MRI-like images using CT images is advantageous for patients who are contraindicated for MRI because of claustrophobia, cardiac pacemakers, and/or artificial joints. Furthermore, imaging using CT is generally less expensive than imaging using MRI. Proposals to achieve CT to MRI translations by combining GANs with dual cycle consistent and voxel-wise losses, show very promising results in brain imaging. An adversarial domain adaptation-based DL approach for automatic tumor segmentation from T2-weighted MRI has been used. Since labelled MRI data for tumor segmentation is scarce, a tumor-aware unsupervised cross-domain adaptation from CT to MRI was used, followed by semi-supervised tumor segmentation using U-Net models trained with synthesized and a limited number of original MRIs.

An emerging line of research has as its objective to train a segmentation network for a target imaging modality without having manual labels of the specific modality. As part of this line of research, the SynSeg-Net (synthetic segmentation network) model has been proposed and has showed encouraging performance for two experiments. In a first of these experiments, CT segmentation of a spleen was conducted using a model that was trained using unpaired CT images and MRI images, where only manual annotations of MRI images were available. In a second of these experiments, intracranial volume segmentation in MRI images was conducted using a model that was trained using unpaired MRI images and CT images, where only manual annotations of CTs were available. A similar problem has also been targeted where the annotation can be ameliorated by leveraging an annotation-rich source modality in learning a segmentation model for an annotation-poor target modality. A network architecture known as the diverse data augmentation generative adversarial network (DDA-GAN) was proposed and showed the effectiveness of this approach in two experiments. In a first of these experiments, segmentation of craniomaxillofacial bony structures was conducted with MRI as the target domain and CT as the source domain containing the manual segmentations. In a second of these experiments, cardiac substructure segmentation with CT as the target domain and MRI as the source domain containing the manual annotations was conducted.

Additional research and development has focused on the automatic segmentation of cartilage in MRI images.

It is known to employ a contrast agent for use when capturing CT images, with the contrast agent aiding with improving contrast of cartilage in CT images thereby aiding with cartilage segmentation. In particular, CT with contrast refers to the administration of a contrast agent or dye during a CT scan. A contrast agent is a substance that enhances the visibility and differentiation of certain tissues and blood vessels, allowing for a more detailed evaluation of specific anatomical structures or pathological conditions. There are drawbacks to the use of contrast agents. These include risks of contrast-related reactions, increased imaging appointment duration, potential kidney damage, potential allergies, and others. A decision to embark on contrast-enhanced CT imaging is made based on the specific clinical scenario, weighing the benefits against potential risks.

Approaches to using MR images and CT images of the same knee may include registration of the MR images and CT images in which information from both modalities is aligned and merged to create a comprehensive and integrated view of the anatomical structures being imaged. Known work focuses on registering MR images and CT images of an individual's knee, after manual segmentation of bone and cartilage has been done, for automatic evaluation of osteoarthritis (OA).

It has also been proposed to evaluate OA in CT instead of in MRI, on the theory that, because OA also affects the trabecular bone, joint space narrowing, and causes subchondral cysts and bone sclerosis, and because these aspects can be very well imaged using the CT modality, OA can be accurately evaluated using CT alone.

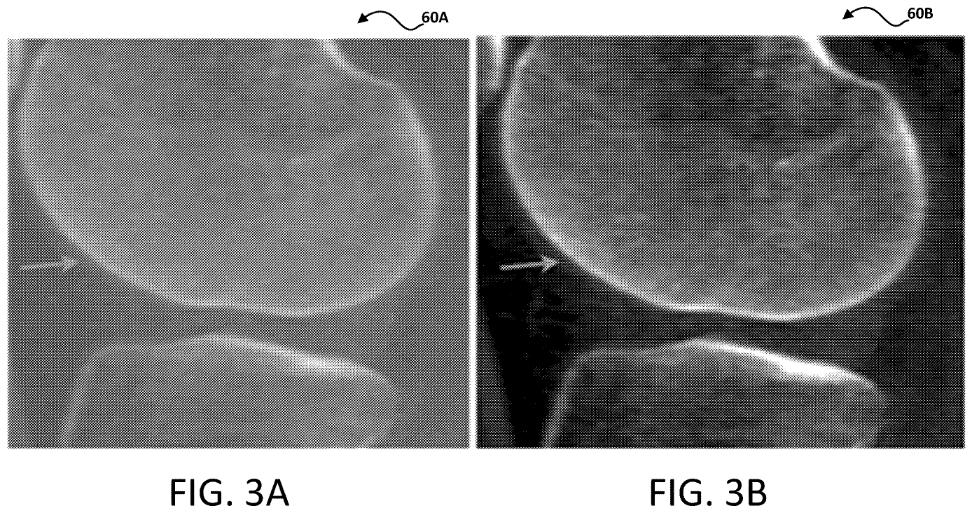

Also, while cartilage itself cannot be easily visualized by a human in a knee CT, it is known from the literature that some bone features in CT images may provide clues that may be used to infer the structure of the cartilage. Also, using a windowing technique, it is possible to change the local contrast of a CT image to better highlight some cartilage structures. FIG. 3A shows an example CT image 60A of a knee joint of an individual human patient without contrast processing, with an arrow pointing to femoral cartilage. FIG. 3B shows the example contrast-processed CT image 60B corresponding to the CT image 60A of FIG. 3A, with contrast processing having been conducted using a local windowing technique, with an arrow pointing to the femoral cartilage. The local windowing technique involves locally manipulating the contrast of the CT image intensity, thereby potentially improving visualization of the cartilage in certain regions.

However, even with local window contrasting, manually annotating the cartilage of a knee CT scan is possible only for certain anatomical locations. As such, it is generally the case that the complete cartilage cannot be segmented by manual annotation alone. Furthermore, local windowing is both: time consuming because different contrasting is required on a per-region basis, and error prone because the cartilage boundary is still, in general, not well-defined.

Provided herein are methods and systems that address the drawbacks of prior techniques by improving training of neural networks for accurately labeling at least soft-tissue such as cartilage in CT images captured of anatomical structures.

### Generating Training Set of Soft-Tissue-Labeled CT Images Using Pairs of MR Images and CT Images of the Same Individual's Anatomy, and Training Using the Generated Training Set.

In a first method, pairs of image sets for each of a plurality of individuals, each pair including both a CT image set and an MR image set of an individual's anatomy, are processed to generate respective three-dimensional (3D) models of the individual's anatomy each including bone of the individual's anatomy. For each individual, the content labeled as bones in both of the resulting pairs of 3D models are aligned/registered thereby to generate a 3D transformation between the bones in the 3D models. Soft-tissue in at least one of the CT images is then labeled based on an application of the 3D transformation to soft-tissue that had been labeled in the 3D model that was generated using the MR image set, in order to create a corresponding soft-tissue-labeled CT image. A training set of labeled CT images is then augmented using the soft-tissue-labeled CT image. For example, the soft-tissue-labeled CT image may be simply added to the training set. Using the training set created in this manner using each of the actual CT and MR image sets for the plurality of individuals, a neural network is trained to label the soft-tissue in CT images. According to this method, therefore, hard-tissue that has been labeled as such in the two different imaging modalities of the same anatomical structure of the same individual provides a linkage between the two modalities that can be used to accurately “transfer” (i.e. transform) labels of soft-tissue from a first modality in which soft-tissue can be accurately labeled, to a second modality in which such soft-tissue cannot generally be as accurately labeled, thereby to generate a training set of accurately soft-tissue-labeled images of the second modality for use in training a neural network to label soft-tissue in images of the second modality.

When training a DL system to segment images, a supervised strategy is typically used. A supervised strategy is one in which a training dataset (or “training set”) contains images with the corresponding segmentation masks. That is, each image in a training set has an associated mask which assigns to each pixel a particular label of interest. These label masks have typically been obtained through manual annotation by experts.

According to this approach, in the CT image sets (which contain multiple CT images each taken along a respective cross-section, or slice, of the anatomy), only the bone labels are manually annotated, while in the MR image sets (which contain multiple MR images each taken along a respective cross-section, or slice, of the anatomy) both bone and cartilage regions are manually annotated. Since the same anatomy (for example, the same knee of the same individual) is imaged using both modalities, 3D models of bone from each modality should, at least ideally, be identical. In an example, the 3D models of the bone may themselves be respectively generated by applying a Marching Cubes process to the contents of the CT image set and to the contents of the MR image set. Once the 3D bone models—a first 3D model of both bone and soft-tissue created using the MR image set, and a second 3D model of bone created using the CT image set—have been generated, the bone of the 3D models may be registered/aligned in order to generate a 3D transformation T between them. This 3D transformation is then available to be applied to the soft-tissue in the first 3D model to label contents in the second 3D model as the same soft-tissue thereby to create an augmented second 3D model. The at least one soft-tissue-labeled CT image may then be created using the augmented second 3D model.

The alignment of the bone in the first and second 3D models may be referred to as 3D registration. In an example, 3D registration proceeds in two steps. In the first of the two steps, known as global registration, a global registration algorithm is applied to obtain an initial alignment between the bones of the first and second 3D models. In an example, an approach known as the 2PNS (2-Point-Normal Sets) process determines this initial alignment. In a second of the two steps, known as alignment refinement, the initial alignment is refined. In an example, an approach known as the iterative closest point (ICP) process is used for alignment refinement, and iteratively refines the alignment obtained during initial alignment. The process of 3D registration produces the 3D transformation T, which can thereafter be applied to the 3D cartilage in the first 3D model to determine the position of cartilage in the second 3D model. In an example, the cartilage labels are then obtained using a Slicer3D process, by resampling the 3D model using the second 3D model (that produced by the CT image set) as the reference volume.

By applying this approach to multiple CT-MR image set pairs, a training set of soft-tissue-labeled CT images is generated and used to train an automatic DL segmentation model (i.e. a trained neural network) so that it is competent to soft-tissue-label CT images of the anatomy. More particularly, the neural network trained according to this approach may be provided with, and thus receive, an input CT image and the neural network may in turn provide a soft-tissue-labeling of the input CT image. The format of the labeling may vary according to implementation. For example, the trained neural network may provide just the labeling to be applied by downstream processes to the pixels of the input CT image, or may provide an entire output CT image that corresponds exactly to the input CT image but additionally has at least the generated soft-tissue labeling integrated with the CT image file in some manner.

In an example, the neural network is a U-net neural network.

Experimental results of the automatic cartilage segmentation system, trained as described herein using training sets created from multiple pairs of image sets of respective individual's anatomy, are compared with a ground truth using two metrics, as shown herein. In this example, the ground truth is defined as the model of bone and cartilage obtained from CT where the cartilage label is determined by aligning the CT with an MRI of the same patient using the bone surface as reference. It will be appreciated that the ground truth itself can be subject to small errors arising from errors in registration and manual labelling of the MR images themselves.

The first of the two metrics, known as the Global Average Symmetric Surface Distance (GASSD), measures the average symmetric surface distance, in millimeters, between the ground-truth surface (that is, the model of bone and cartilage where the cartilage label is obtained by registration with an MR image as explained herein) and the reconstructed surface from the automatic segmentation. For each point in the reference surface, the closest triangle in the other surface is determined. This is performed using both the ground-truth surface and the reconstructed surface as reference. The average of these distances is then computed, with lower values corresponding to better segmentation.

The second of the two metrics, known as Cartilage Average Symmetric Surface Distance (CASSD), measures the average symmetric surface distance, in millimeters, between the ground-truth surface and the reconstructed surface only in the points where cartilage is typically located. For example, at the condyles of the femur and the plateaus of the tibia). Lower values correspond to better segmentation.

Table 1, below, provides an overview of two different sets of pairs of CT and MRI image sets used to generate training datasets for training two different neural networks, and test datasets used to test the respective neural networks.

TABLE 1

| | | | Size | | | |
|---|---|---|---|---|---|---|
| | | | of | Size | | |
| | | Im- | train- | of | | |
| | | aged | ing | test | | |
| Data- | | ana- | data- | data- | CT | MRI |
| sets | Origin | tomy | set | set | Specs | Specs |
| Data | PRIME | Cad- | 10 | 2 | Standard- | PRIME |
| set 1 | Bioskills | aver | scans | scans | of-care | Se- |
| | Labs | spec- | | | | quence |
| | | imen | | | | |
| Data | Standard- | Liv- | 70 | 19 | Standard- | Standard- |
| set 2 | of-care, | ing | scans | scans | of-care | of-care |
| | US | hu- | | | | (PD Sag |
| | | man | | | | no FS) |

Table 1.

In particular, two different DL models were developed. The first of these, referred to herein as model_Bioskills, was trained with the training set of dataset 1. The second of these, referred to herein as model_SoC, was trained with the training dataset of dataset 2. PD Sag no FS refers to Proton Density Sagittal no Fat Saturation. More particularly, a proton density (PD) MRI sequence is a specific MRI imaging sequence largely used in SoC, Sag is sagittal and concerns the acquisition direction, and no FS is no Fat Saturation, meaning that a no fat suppression technique was employed during the MRI.

The experimental results of the two DL models in the two datasets detailed in Table 1 are presented in Table 2, below.

TABLE 2

| Auto- | | Num- | | | | |
|---|---|---|---|---|---|---|
| matic | | ber | | | | |
| segmen- | Test | of | Femur | Femur | Tibia | Tibia |
| tation | data- | test | GASSD | CASSD | GASSD | CASSD |
| model | set | scans | (mm) | (mm) | (mm) | (mm) |
| model_ | Data- | 2 | 0.39 | 0.73 | 0.27 | 0.64 |
| Bioskills | set 1 | | | | | |
| model _ | Data- | 19 | 0.45 | 1.08 | 0.24 | 0.57 |
| Bioskills | set 2 | | | | | |
| model_ | Data- | 2 | 0.73 | 1.07 | 0.79 | 0.77 |
| SoC | set 1 | | | | | |
| model_ | Data- | 19 | 0.33 | 0.72 | 0.19 | 0.46 |
| SoC | set 2 | | | | | |

In Table 2, median GASSD and CASSD between ground truth and inferred models is shown.

For quantifying the difference of a ground-truth bone model with respect to a ground-truth model containing both bone and cartilage, in median terms, it was found that the difference in CASSD is +/−0.5 mm.

In the example described herein, the registration of the bone in first and second 3D models created using MR image sets and CT image sets may be used to generate a 3D transformation useful for transferring soft-tissue-labeling from MR images to CT images for use in generating a training set of soft-tissue-labeled CT images. In another example, a similar approach conducts registration/alignment of pairs of CT and MR images of respective individuals' anatomy to generate a 2D transformation between bone in the MR image of the pair and the CT image of the pair. This 2D transformation may then be applied to the labeled soft-tissue in the MR image to label soft-tissue in the CT image, thereby to create a soft-tissue-labeled CT image. This may be done individually for multiple CT-MR image pairs taken from respective CT and MR image sets of the individuals' anatomy, thereby producing a 2D transformation and thus a soft-tissue-labeled CT image for each CT-MR image pair. With this approach, registration/alignment is done as between the bone in 2D CT and MR images, rather than as between the bone in 3D CT and MR bone models.

The alignment of the bone in the CT and MR images may be referred to as 2D registration. In an example, 2D registration proceeds in two steps. In the first of the two steps, known as global registration, a global registration algorithm is applied to obtain an initial alignment between the bones of the CT and MR images. In an example, an approach known as the 2PNS (2-Point-Normal Sets) process determines this initial alignment. In a second of the two steps, known as alignment refinement, the initial alignment is refined. In an example, an approach known as the iterative closest point (ICP) process is used for alignment refinement, and iteratively refines the alignment obtained during initial alignment. The process of 2D registration produces the 2D transformation T2, which can thereafter be applied to the 2D cartilage in the MR image to determine the position of cartilage in the CT image with which it is paired.

By applying this approach to multiple CT-MR image pairs, a training set of soft-tissue-labeled CT images is generated and used to train an automatic DL segmentation model (i.e. a trained neural network) so that it is competent to soft-tissue-label CT images of the anatomy. More particularly, the neural network trained according to this approach may be provided with, and thus receive, an input CT image and the neural network may in turn provide a soft-tissue-labeling of the input CT image. The format of the labeling may vary according to implementation. For example, the trained neural network may provide just the labeling to be applied by downstream processes to the pixels of the input CT image, or may provide an entire output CT image that corresponds exactly to the input CT image but additionally has at least the generated soft-tissue labeling integrated with the CT image file in some manner.

In an example, the neural network is a U-net neural network.

### Generating Training Set of Soft-Tissue-Labeled CT Images by Transferring Soft-Tissue Labeling of Synthetically-Generated MR Images to Input CT Images.

As it can be costly and time-consuming to capture and/or obtain actual captured pairs of CT and MR image sets of the same individual's anatomy, alternatives approaches for soft-tissue labeling CT images are of interest.

In the following example, a neural network is trained using a generative adversarial approach to receive CT images of individuals' anatomy and to generate respective output synthetic MR images of the individuals' anatomy, with the output synthetic MR images being usefully-accurate such that they can be used for MRI segmenting in lieu of actual MR images captured using MRI technology.

In particular the neural network receives an input CT image and produces a corresponding output synthetic MR image. This output synthetic MR image is thereafter processed for soft-tissue-labeling. Such processing for soft-tissue labeling may be conducted by an automatic image segmentation system, which can be configured to label soft-tissue and hard-tissue in MR images that it is provided to process. At least the soft-tissue labeling can then be applied to the input CT image used to produce the synthetic output MR image thereby to create a soft-tissue-labeled CT image.

To become competent to generate output synthetic MR images corresponding to input CT images, in this example the neural network is configured as a first generative adversarial network (GAN) interconnected with a second GAN in a CycleGAN configuration for training. In this example, the first GAN is itself trained to generate a synthetic output MR image from a first GAN input, and the second GAN is itself trained to generate a synthetic CT image from a second GAN input. The overall neural network is trained by, for each of plurality of cycles, conducting a number of steps. A first of these includes providing, as a first GAN input, a synthetic CT image generated by the second GAN thereby to generate, as a first GAN output, a corresponding synthetic MR image. A second of these steps includes providing, as a second GAN input, a synthetic MR image generated by the first GAN thereby to generate, as a second GAN output, a corresponding synthetic CT image. A third of these steps includes determining a first measure of difference between the second GAN output and the first GAN input. A fourth of these steps includes determining a second measure of difference between the first GAN output and the second GAN input. A fifth of these steps includes modifying the first GAN and the second GAN based at least on the first measure of difference and the second measure of difference. The modifying is conducted to reduce, over successive cycles, magnitudes of the first measure of difference and the second measure of difference.

This CycleGAN approach conducted over multiple cycles, causes the neural network to converge on cycle consistency, such that it becomes competent to generate usefully-accurate output synthetic MR images based on input actual CT images, including the soft-tissue that would be present in an MR image were it actually captured of the anatomy using MRI technology. It will be appreciated that, by virtue of the CycleGAN architecture and approach, the training of the neural network can be done without the respective actual CT and actual MR images used for training the first and second GANs themselves having to be pairs captured of the same actual individuals' anatomy.

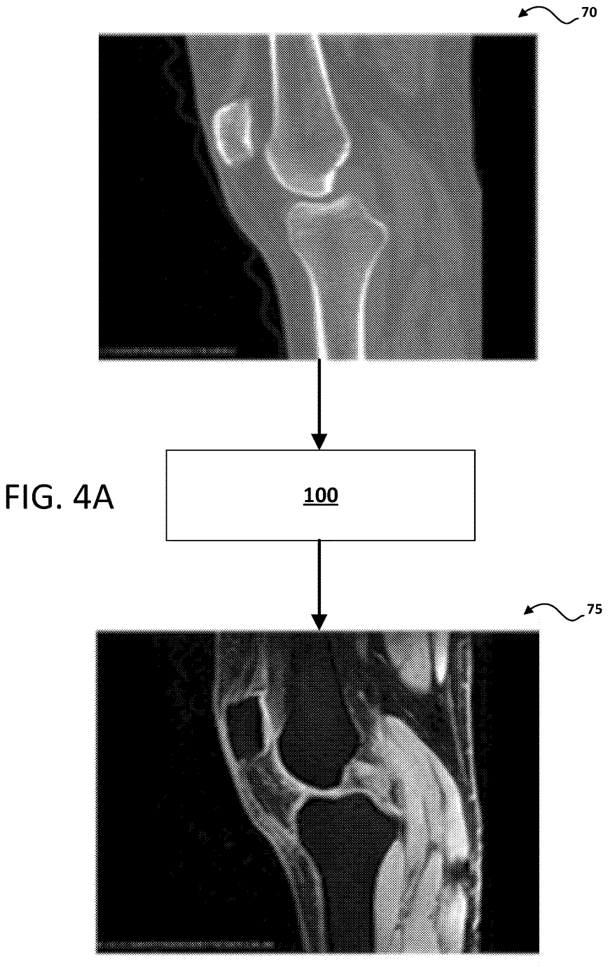

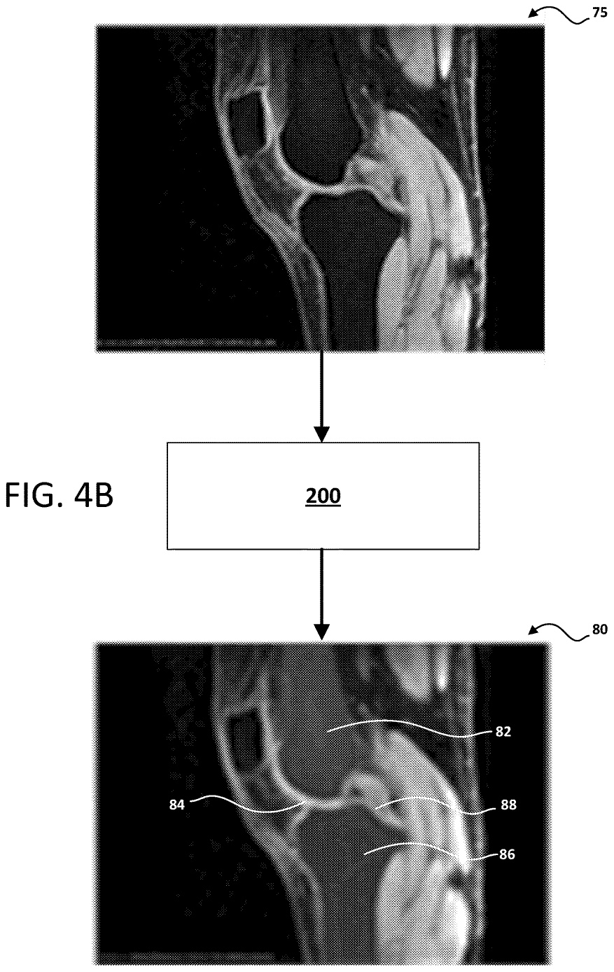

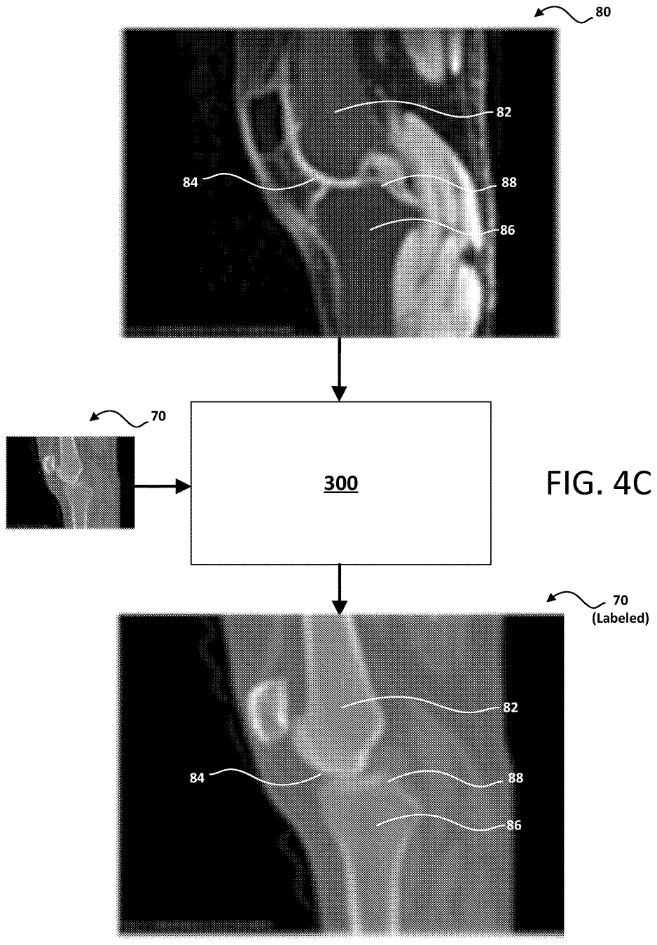

FIGS. 4A, 4B, and 4C show an overall example pipeline that uses the neural network capable of generating a synthetic MR image from an input CT image. In FIG. 4A, the neural network, identified with reference numeral 100, receives a real, unlabeled CT image 70 and generates a synthetic MR image 75. In FIG. 4B, the synthetic MR image 75 generated by neural network 100 is provided to an automatic MRI segmentation system 200, which in this example infers soft-tissue such as cartilage and hard-tissue such as bone to generate soft-tissue and hard-tissue labeling thereby to create a labeled synthetic MR image 80. In this example, the automatic MRI segmentation system 200 is used for inferring, and thus generating labels for, femur bone 82, femoral cartilage 84, tibia bone 86 and tibial cartilage 88. Finally, the inferred labels are transferred by a resampling process 300 to the CT image using a resampling technique of Slicer3D, with the CT image as the reference volume. It will be appreciated that the segmentation of hard-tissue such as bone does not have to be done directly on the input CT image, as the labels generated by the resampling process 300 for the synthetic MR image for both hard-tissue and soft-tissue may be transferred, using resampling, to the CT image. The CT image 70 thus is provided with labels for the femur bone 82, femoral cartilage 84, tibia bone 86 and tibial cartilage 88.

The experimental results for the pipeline carried out according to FIGS. 4A, 4B, and 4C, are shown in Table 3, below. Experimental results of the proposed solution that combines the CycleGAN and the automatic PRIME MRI segmentation algorithm are shown. Median GASSD and CASSD between ground truth and inferred models are shown.

TABLE 3

| | Number | Femur | Femur | Tibia | Tibia |
|---|---|---|---|---|---|
| Test | of test | GASSD | CASSD | GASSD | CASSD |
| dataset | scans | (mm) | (mm) | (mm) | (mm) |
| Dataset 1 | 2 | 1.33 | 1.48 | 1.66 | 1.09 |
| Dataset 2 | 19 | 0.36 | 0.86 | 0.15 | 0.51 |

In another example, during training of the neural network, bone segmentation/labeling for the synthetic CT image generated by the second GAN is received and used as regularization during the modifying. More particularly, the neural network is configured as a synthetic segmentation network (SynSeg-Net) to train to generate the synthetic MR image using the bone CT segmentation as regularization. It was found, during testing of the SynSeg-Net configuration that the additional segmentation loss improved the quality of the generated synthetic images. The experimental results, including median GASSD and CASSD between ground truth and the inferred models, are shown in Table 4, below.

TABLE 4

| | Number | Femur | Femur | Tibia | Tibia |
|---|---|---|---|---|---|
| Test | of test | GASSD | CASSD | GASSD | CASSD |
| dataset | scans | (mm) | (mm) | (mm) | (mm) |
| Dataset 1 | 2 | 1.33 | 1.48 | 1.66 | 1.09 |
| Dataset 2 | 19 | 0.33 | 0.83 | 0.95 | 0.77 |

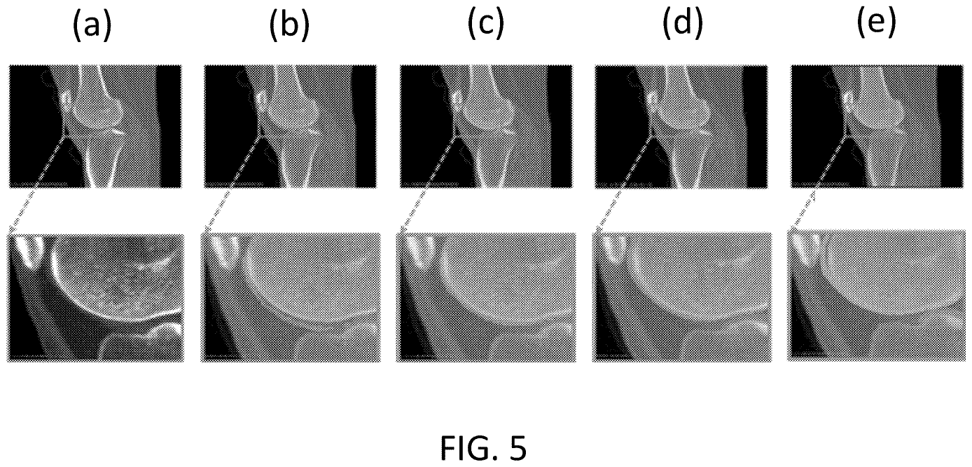

FIG. 5 shows various CT and CT enlarged/zoomed-in images illustrative of comparison of (a) an input CT image with (b) a pseudo ground-truth segmentation; (c) an automatic segmentation using the image pair approach described herein; (d) an automatic segmentation using the CycleGAN approach described herein; and (e) an automatic segmentation using the SynSeg-Net approach described herein. It will be appreciated that the ground-truth for cartilage segmentation obtained from MRI include some errors due, for example, to difficulty with manual cartilage delineation, registration inaccuracies, or other causes.

To account for the errors, Table 5 and Table 6 present preliminary results showing the performance of automatic segmentation of cartilage in CT images for intra-operative registration using the PRIME (now referred-to as TESSA) application provided by Smith & Nephew of Andover, MA.

TABLE 5

| | Median | Median |
|---|---|---|
| | FP error | TL error |
| Imaging Modality | (mm) | (mm) |
| CT Bone | 1.85 | 1.45 |
| PRIME MRI Bone | 2.64 | 1.64 |
| PRIME MRI Bone + Cartilage | 2.00 | 1.50 |
| CT Bone + Cartilage (GAN) | 1.59 | 1.45 |
| CT Bone + Cartilage | 2.47 | 1.87 |
| (CT-MRI Pairs SoC) | | |
| CT Bone + Cartilage | 1.96 | 1.94 |
| (CT-MRI Pairs BioSkills) | | |
| CT Bone + Cartilage (SynSeg) | 2.35 | 1.67 |

In Table 5, results of simulation of PRIME intra-op registration using 3D models from different sources (1 ACL femur case) are shown. Normal intra-operative data acquisition was performed where most of the data corresponds to bone and some to cartilage. [CT Bone] refers to automatic bone segmentation of CT, [CT Bone+Cartilage (GAN)] refers to automatic segmentation of bone and cartilage in CT using the CycleGAN approach described herein, and [CT Bone+Cartilage (SynSeg)] refers to automatic segmentation of bone and cartilage in CT using the SynSeg-Net modified approach described herein. [CT Bone+Cartilage (CT-MRI Pairs SoC)] refers to automatic segmentation of bone and cartilage in CT using model_SoC, [CT Bone+Cartilage (CT-MRI Pairs BioSkills)] refers to automatic segmentation of bone and cartilage in CT using model_bioskills, [PRIME MRI Bone] refers to automatic segmentation of bone in PRIME MRI, and [PRIME MRI Bone+Cartilage] refers to automatic segmentation of bone and cartilage in PRIME MRI. Downward hash shading, grid hash shading, and upward hash shading corresponds, respectively, to best, second best and third best metric in a given column.

TABLE 6

| | Median | Median |
|---|---|---|
| | FP error | TL error |
| Imaging Modality | (mm) | (mm) |
| CT Bone | 2.77 | 1.09 |
| PRIME MRI Bone | 2.60 | 1.15 |
| PRIME MRI Bone + Cartilage | 1.64 | 1.00 |
| CT Bone + Cartilage (GAN) | 1.52 | 1.59 |
| CT Bone + Cartilage | 2.56 | 2.28 |
| (CT-MRI Pairs SoC) | | |
| CT Bone + Cartilage | 2.30 | 1.56 |
| (CT-MRI Pairs BioSkills) | | |
| CT Bone + Cartilage (SynSeg) | 1.91 | 1.08 |

In Table 6, results of simulation of PRIME intra-op registration using 3D models from different sources, is shown. Intra-operative data acquisition was performed acquiring both bone and cartilage points (1 ACL femur case). [CT Bone] refers to automatic bone segmentation of CT, [CT Bone+Cartilage (GAN)] refers to automatic segmentation of bone and cartilage in CT using the CycleGAN approach described herein, [CT Bone+Cartilage (SynSeg)] refers to automatic segmentation of bone and cartilage in CT using the SynSeg-Net modification, [CT Bone+Cartilage (CT-MRI Pairs SoC)] refers to automatic segmentation of bone and cartilage in CT using model_SoC, [CT Bone+Cartilage (CT-MRI Pairs BioSkills)] refers to automatic segmentation of bone and cartilage in CT using model_bioskills, [PRIME MRI Bone] refers to automatic segmentation of bone in PRIME MRI, and [PRIME MRI Bone+Cartilage] refers to automatic segmentation of bone and cartilage in PRIME MRI. Downward hash shading, grid hash shading, and upward hash shading corresponds, respectively, to best, second best and third best metric in a given column. Green, blue and yellow corresponds, respectively, to best, second best and thirst best metric in column.

As shown in FIG. 5, the ground-truth segmentation shown in the CT images at (b), obtained from MRI, has some errors. That is, the cartilage is not correctly aligned with bone as might be expected. In contrast, the automatic segmentation approaches shown in the CT images at (c), (d), and (e), provided more plausible results. These results were discussed with a radiologist who studied the results of the automatic segmentations shown at (c), (d), and (e), and regarded them as reasonable, though subject to improvement in certain reasons, and difficult to evaluate quantitatively.

The various approaches to segmentation of CT images described herein provide a number of advantages. For example, by enabling CT images to be soft-tissue labeled, 3D modeling of the anatomical structures of a given joint or other anatomy can be enhanced to include not just hard-tissue such as the bone of the joint, but soft-tissue such as the cartilage of the joint. Intra-operative digitization of the joint so that it can be registered with a three-dimensional model of the joint for surgical planning and navigation can thereby be done using structures in the joint whether hard-tissue such as bone or soft-tissue such as cartilage. Because preliminary studies have shown (1) that it is very difficult to only digitize bone, and the surgeon sometimes also digitizes some cartilage structures (as though it were bone), and (2) that digitizing cartilage intra-operatively and using 3D models that not only represent bone but also include cartilage improves the registration results, it is expected that providing 3D models of the anatomy that represent both bone and cartilage can improve overall performance of the surgical navigation. It is the case that, as at the time of this application, 3D models composed of bone and cartilage can generally only be obtained from MRI imaging, because, as discussed herein, cartilage has not heretofore been satisfactorily visualized in CT. As the techniques described herein permit the inference and corresponding labeling of cartilage using just CT images, it may be possible for surgical navigation solutions, such as that provided by the PRIME ACL solution offered by Smith & Nephew of Andover, MA, U.S.A., to use standard-of-care CT to obtain 3D models of both bone and cartilage and/or of other kinds of soft-tissue.

The current standard for analyzing and diagnosing cartilage diseases, including osteoarthritis (OA), is to use MRI technology. The various approaches provided herein may be useful for helping with studies of OA progress using CT images, by considering how the OA affects the trabecular bone, joint space narrowing, and/or causes subchondral cysts and bone sclerosis.

### Software and Hardware

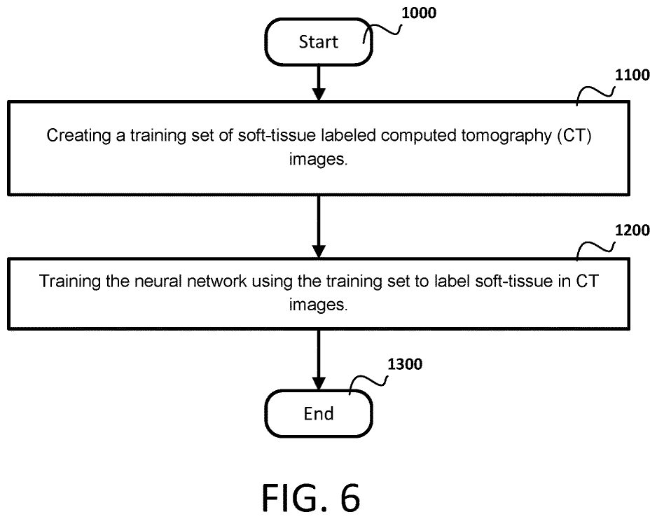

FIG. 6 shows a computer-implemented method of training a neural network for soft-tissue labeling, in accordance with at least some examples. In particular, the method starts (block 1000) and comprises: creating a training set of soft-tissue labeled computed tomography (CT) images (block 1100), and training the neural network using the training set to label soft-tissue in CT images (block 1200). Thereafter, the method ends (block 1300).

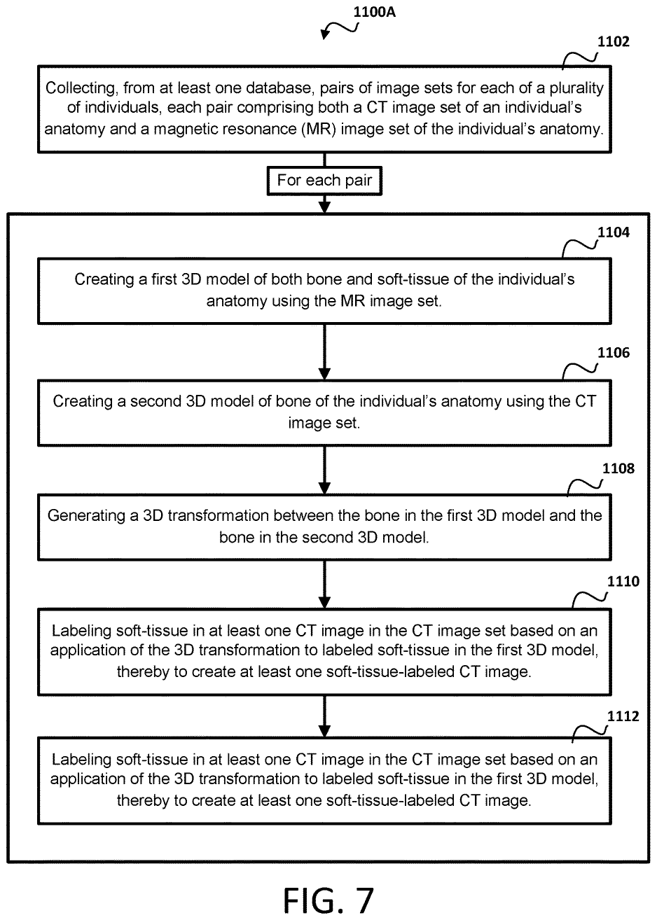

FIG. 7 shows steps of the method that may be conducted during creating the training set of soft-tissue labeled CT images at block 1100 in FIG. 6, according to an example 1100A. In particular, these steps comprise: collecting, from at least one database, pairs of image sets for each of a plurality of individuals, each pair comprising both a CT image set of an individual's anatomy and a magnetic resonance (MR) image set of the individual's anatomy (block 1102) and, for each of the pairs: creating a first 3D model of both bone and soft-tissue of the individual's anatomy using the MR image set (block 1104), creating a second 3D model of bone of the individual's anatomy using the CT image set (block 1106), generating a 3D transformation between the bone in the first 3D model and the bone in the second 3D model (block 1108), labeling soft-tissue in at least one CT image in the CT image set based on an application of the 3D transformation to labeled soft-tissue in the first 3D model, thereby to create at least one soft-tissue-labeled CT image (block 1110); and augmenting the training set with the at least one soft-tissue-labeled CT image (block 1112).

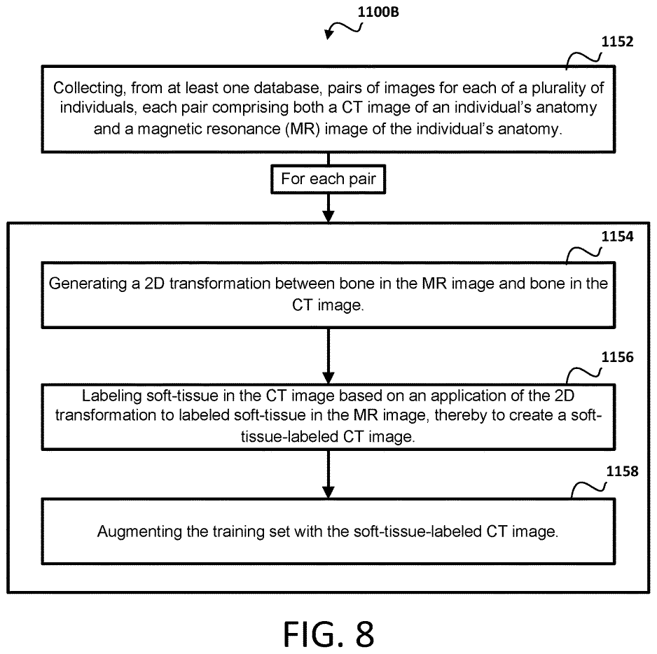

FIG. 8 shows alternative steps of the method that may be conducted during creating the training set of soft-tissue labeled CT images at block 1100 in FIG. 6, according to another example. In this example, 2D transformations between pairs of CT and MR images are conducted, rather than 3D transformations between bones in first and second 3D models. In particular, these steps comprise: collecting, from at least one database, pairs of images for each of a plurality of individuals, each pair comprising both a CT image of an individual's anatomy and a magnetic resonance (MR) image of the individual's anatomy (block 1152) and, for each of the pairs: generating a 2D transformation between bone in the MR image and bone in the CT image (block 1154), labeling soft-tissue in the CT image based on an application of the 2D transformation to labeled soft-tissue in the MR image, thereby to create a soft-tissue-labeled CT image (block 1156), and augmenting the training set with the soft-tissue-labeled CT image (block 1158).

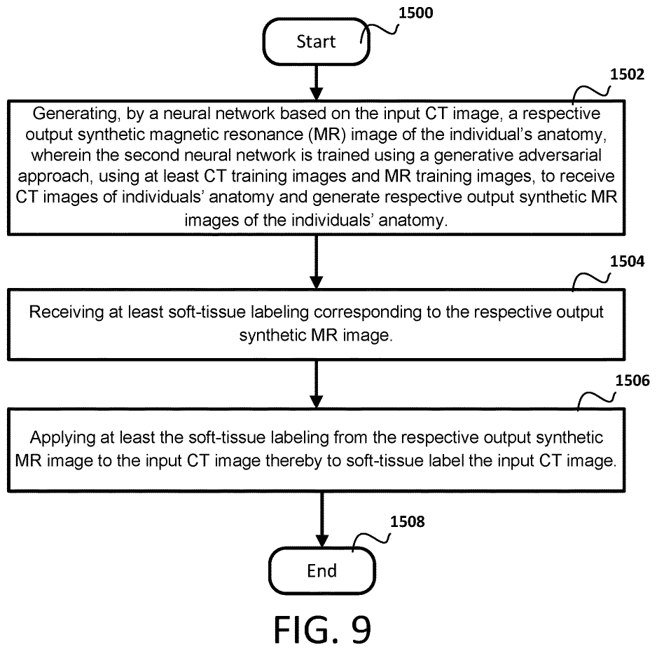

FIG. 9 shows a computer-implemented method of soft-tissue labeling an input CT image of an individual's anatomy, in accordance with an example. In particular, the method starts (block 1500) and comprises: generating, by a neural network based on the input CT image, a respective output synthetic magnetic resonance (MR) image of the individual's anatomy, wherein the neural network is trained using a generative adversarial approach, using at least CT training images and MR training images, to receive CT images of individuals' anatomy and generate respective output synthetic MR images of the individuals' anatomy (block 1502); receiving at least soft-tissue labeling corresponding to the respective output synthetic MR image (block 1504); and applying at least the soft-tissue labeling from the respective output synthetic MR image to the input CT image thereby to soft-tissue label the input CT image (block 1506).

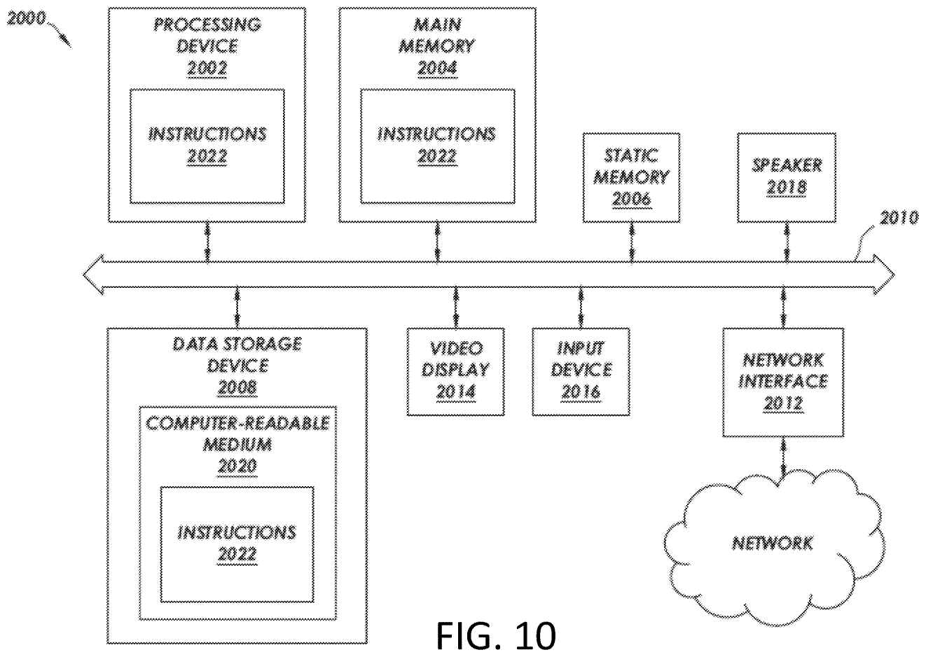

FIG. 10 shows an example computer system 2000 that may be configured to execute the processor-implemented methods disclosed herein, and that may be configured to implement the systems disclosed herein. In one example, computer system 2000 may correspond to a surgical controller, a separate computing device, or any other system that implements any or all the various methods discussed in this specification. The computer system 2000 may be connected (e.g., networked) to other computer systems in a local-area network (LAN), an intranet, and/or an extranet (e.g., device cart 402 network), or at certain times the Internet (e.g., when not in use in a surgical procedure). The computer system 2000 may be a server, a personal computer (PC), a tablet computer or any device capable of executing a set of instructions (sequential or otherwise) that specify actions to be taken by that device. Further, while only a single computer system is illustrated, the term “computer” shall also be taken to include any collection of computers that individually or jointly execute a set (or multiple sets) of instructions to perform any one or more of the methods discussed herein.

The computer system 2000 includes a processing device 2002, a main memory 2004 (e.g., read-only memory (ROM), flash memory, dynamic random access memory (DRAM) such as synchronous DRAM (SDRAM)), a static memory 2006 (e.g., flash memory, static random access memory (SRAM)), and a data storage device 2008, which communicate with each other via a bus 2010.

Processing device 2002 represents one or more general-purpose processing devices such as a microprocessor, central processing unit, or the like. More particularly, the processing device 2002 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device 2002 may also be one or more special-purpose processing devices such as an application specific integrated circuit (ASIC), a field programmable gate array (FPGA), a digital signal processor (DSP), network processor, or the like. The processing device 2002 is configured to execute instructions for performing any of the operations and steps discussed herein. Once programmed with specific instructions, the processing device 2002, and thus the entire computer system 2000, becomes a special-purpose device, such as the surgical controller 418.

The computer system 2000 may further include a network interface device 2012 for communicating with any suitable network (e.g., the device cart 402 network). The computer system 2000 also may include a video display 2014 (e.g., display device 414), one or more input devices 2016 (e.g., a microphone, a keyboard, and/or a mouse), and one or more speakers 2018. In one illustrative example, the video display 2014 and the input device(s) 2016 may be combined into a single component or device (e.g., an LCD—liquid crystal display—touch screen).

The data storage device 2008 may include a computer-readable storage medium 2020 serving as memory on which the instructions 2022 (e.g., implementing any methods and any functions performed by any device and/or component depicted described herein) embodying any one or more of the methodologies or functions described herein is stored. The instructions 2022 may also reside, completely or at least partially, within the main memory 2004 and/or within the processing device 2002 during execution thereof by the computer system 2000. As such, the main memory 2004 and the processing device 2002 also constitute computer-readable media. In certain cases, the instructions 2022 may further be transmitted or received over a network via the network interface device 2012.

While the computer-readable storage medium 2020 is shown in the illustrative examples to be a single medium, the term “computer-readable storage medium” or “processor-readable storage medium” should be taken to include a single medium or multiple media (e.g., a centralized or distributed database, and/or associated caches and servers) that store the one or more sets of instructions. The term “computer-readable storage medium” or “processor-readable storage medium” shall also be taken to include any medium that is capable of storing, encoding or carrying a set of instructions for execution by the machine and that cause the machine to perform any one or more of the methodologies of the present disclosure. The term “computer-readable storage medium” or “processor-readable storage medium” shall accordingly be taken to include, but not be limited to, solid-state memories, optical media, and magnetic media.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

For example, while examples herein pertain to cartilage segmentation in knee CT images, the methods and systems described and depicted herein may be applied to infer the segmentation of other musculoskeletal (MSK) soft-tissue anatomical structures that can be well-perceived in MRI and not in CT. This may include the cartilage of the examples herein, but also other soft tissues such as the Anterior Cruciate Ligament (ACL), Posterior Cruciate Ligament (PCL), menisci, muscles, tendons, medial collateral ligament (MCL), lateral collateral ligament (LCL), and others.

### CLAUSES

Clause 1. A processor-implemented method of training a neural network for soft-tissue labeling comprising:
creating a training set of soft-tissue labeled computed tomography (CT) images comprising:
collecting, from at least one database, pairs of image sets for each of a plurality of individuals, each pair comprising both a CT image set of an individual's anatomy and a magnetic resonance (MR) image set of the individual's anatomy; and
for each of the pairs:
creating a first 3D model of both bone and soft-tissue of the individual's anatomy using the MR image set;
creating a second 3D model of bone of the individual's anatomy using the CT image set;
generating a 3D transformation between the bone in the first 3D model and the bone in the second 3D model;
labeling soft-tissue in at least one CT image in the CT image set based on an application of the 3D transformation to labeled soft-tissue in the first 3D model, thereby to create at least one soft-tissue-labeled CT image; and
augmenting the training set with the at least one soft-tissue-labeled CT image;
and
training the neural network using the training set to label soft-tissue in CT images.

Clause 2. The processor-implemented method of clause 1, wherein labeling soft-tissue in at least one CT image in the CT image set based on an application of the 3D transformation to labeled soft-tissue in the first 3D model comprises:
applying the 3D transformation to the soft-tissue in the first 3D model to label contents of the second 3D model as soft-tissue thereby to create an augmented second 3D model; and
creating the at least one soft-tissue-labeled CT image using the augmented second 3D model.

Clause 3. The processor-implemented method of clause 1, wherein generating a 3D transformation between the bone in the first 3D model and the bone in the second 3D model comprises:
applying a global registration process to determine an initial alignment transformation between the bone in the first 3D model and the bone in the second 3D model; and
applying an iterative closest point (ICP) process to the initial alignment transformation thereby to generate the 3D transformation.

Clause 4. The processor-implemented method of clause 3, wherein applying a global registration process to determine an initial alignment transformation between the bone in the first 3D model and the bone in the second 3D model comprises applying a 2PNS (2-Point-Normal Sets) process to determine the initial alignment transformation between the bone in the first 3D model and the bone in the second 3D model.

Clause 5. The processor-implemented method of clause 1, wherein the neural network is a U-net neural network.

Clause 6. The processor-implemented method of clause 1, wherein creating a first 3D model of both bone and soft-tissue of the individual's anatomy using the MR image set comprises:
receiving at least bone labeling and soft-tissue labeling corresponding to MR images in the MR image set; and
applying a Marching Cubes process to the bone and soft-tissue labeling of the MR images.

Clause 7. The processor-implemented method of clause 1, wherein creating a second 3D model of bone of the individual's anatomy using the CT image set comprises:
receiving at least bone labeling corresponding to CT images in the CT image set; and
applying a Marching Cubes process to the bone labeling of the CT images.

Clause 8. The processor-implemented method of clause 1, wherein the individual's anatomy is a knee joint, wherein labeling soft-tissue in at least one CT image in the CT image set comprises labeling femoral cartilage and tibial cartilage of the knee joint.

Clause 9. A non-transitory processor-readable medium embodying processor-readable program code executable by at least one processor to carry out the processor-implemented method of clause 1.

Clause 10. A neural network trained according to the processor-implemented method of clause 1.

Clause 11. The neural network of clause 10, wherein the neural network is a U-net neural network.

Clause 12. A processor-implemented method of soft-tissue-labeling a computed tomography (CT) image, the method comprising:
providing an input CT image as input to the neural network of clause 10; and
receiving, from the neural network, a soft-tissue-labeling of the input CT image.

Clause 13. A system for labeling soft-tissue in a computed tomography (CT) image, the system comprising:
at least one processor; and
a memory coupled to the at least one processor, the memory storing instructions that, when executed by the at least one processor, cause the at least one processor to:
   provide an input CT image as input to the neural network of clause 10; and
   receive, from the neural network, a soft-tissue-labeling of the input CT image.

Clause 14. The processor-implemented method of clause 1, wherein the soft-tissue is cartilage.

Clause 15. A processor-implemented method of training a neural network for soft-tissue labeling comprising:
creating a training set of soft-tissue-labeled computed tomography (CT) images comprising:
collecting, from at least one database, pairs of images for each of a plurality of individuals, each pair comprising both a CT image of an individual's anatomy and a magnetic resonance (MR) image of the individual's anatomy; and
for each of the pairs:
generating a 2D transformation between bone in the MR image and bone in the CT image;
labeling soft-tissue in the CT image based on an application of the 2D transformation to labeled soft-tissue in the MR image, thereby to create a soft-tissue-labeled CT image; and
augmenting the training set with the soft-tissue-labeled CT image;
and
training the neural network using the training set to label soft-tissue in CT images.

Clause 16. The processor-implemented method of clause 15, wherein labeling soft-tissue in the CT image based on an application of the 2D transformation to labeled soft-tissue in the MR image comprises:
applying the 2D transformation to the soft-tissue in the MR image to label contents of the CT image as soft-tissue thereby to create the soft-tissue-labeled CT image.

Clause 17. The processor-implemented method of clause 15, wherein generating a 2D transformation between the bone in the MR image and the bone in the CT image comprises:
applying a global registration process to determine an initial alignment transformation between the bone in the MR image and the bone in the CT image; and
applying an iterative closest point (ICP) process to the initial alignment transformation thereby to generate the 2D transformation.

Clause 18. The processor-implemented method of clause 15, wherein the neural network is a U-net neural network.

Clause 19. The processor-implemented method of clause 15, wherein the individual's anatomy is a knee joint, wherein labeling soft-tissue in the CT image comprises labeling femoral cartilage and tibial cartilage of the knee joint.

Clause 20. A non-transitory processor-readable medium embodying processor-readable program code executable by at least one processor to carry out the processor-implemented method of clause 15.

Clause 21. A neural network trained according to the processor-implemented method of clause 15.

Clause 22. The neural network of clause 21, wherein the neural network is a U-net neural network.

Clause 23. A processor-implemented method of soft-tissue-labeling a computed tomography (CT) image, the method comprising:
providing an input CT image as input to the neural network of clause 21; and
receiving, from the neural network, a soft-tissue-labeling of the input CT image.

Clause 24. A system for labeling soft-tissue in a computed tomography (CT) image, the system comprising:
at least one processor; and
a memory coupled to the at least one processor, the memory storing instructions that, when executed by the at least one processor, cause the at least one processor to:
   provide an input CT image as input to the neural network of clause 21; and
   receive, from the neural network, a soft-tissue-labeling of the input CT image.

Clause 25. The processor-implemented method of clause 15, wherein the soft-tissue is cartilage.

Clause 26. A processor-implemented method of soft-tissue labeling an input CT image of an individual's anatomy comprising:
generating, by a neural network based on the input CT image, a respective output synthetic magnetic resonance (MR) image of the individual's anatomy, wherein the neural network is trained using a generative adversarial approach, using at least CT training images and MR training images, to receive CT images of individuals' anatomy and generate respective output synthetic MR images of the individuals' anatomy;
receiving at least soft-tissue labeling corresponding to the respective output synthetic MR image; and
applying at least the soft-tissue labeling from the respective output synthetic MR image to the input CT image thereby to soft-tissue label the input CT image.

Clause 27. The processor-implemented method of clause 26, wherein receiving at least soft-tissue labeling corresponding to the output synthetic MR image comprises:
providing the output synthetic MR image to an automatic image segmentation system; and
receiving at least the soft-tissue labeling corresponding to the output synthetic MR image from the automatic image segmentation system.

Clause 28. The processor-implemented method of clause 27, wherein applying at least the soft-tissue labeling from the output synthetic MR image to the input CT image thereby to soft-tissue label the input CT image comprises:
resampling a segmentation volume of the output synthetic MR image using the input CT image as a reference.

Clause 29. The processor-implemented method of clause 26, further comprising:
configuring the neural network as a first generative adversarial network (GAN) interconnected with a second GAN, wherein the first GAN is trained to generate a synthetic output MR image from a first GAN input, and the second GAN is trained to generate a synthetic CT image from a second GAN input; and
training the neural network by, for each of a plurality of cycles:
providing, as a first GAN input, a synthetic CT image generated by the second GAN thereby to generate, as a first GAN output, a corresponding synthetic MR image;
providing, as a second GAN input, a synthetic MR image generated by the first GAN thereby to generate, as a second GAN output, a corresponding synthetic CT image;
determining a first measure of difference between the second GAN output and the first GAN input;
determining a second measure of difference between the first GAN output and the second GAN input; and
modifying the first GAN and the second GAN based at least on the first measure of difference and the second measure of difference, wherein the modifying is conducted to reduce, over successive cycles, magnitudes of the first measure of difference and the second measure of difference.

Clause 30. The processor-implemented method of clause 29, further comprising:
during training of the neural network:
receiving bone labeling for the synthetic CT image generated by the second GAN; and
using the bone labeling as regularization during the modifying.

Clause 31. The processor-implemented method of clause 26, wherein the neural network is a U-net neural network.

Clause 32. The processor-implemented method of clause 26, wherein the individual's anatomy is a knee joint and the soft-tissue-labeling includes labeling for femoral cartilage and labeling for tibial cartilage.

Clause 33. A non-transitory processor-readable medium embodying processor-readable program code executable by at least one processor to carry out the processor-implemented method of clause 26.

Clause 34. A neural network trained according to the processor-implemented method of clause 26.

Clause 35. The neural network of clause 34, wherein the neural network is a U-net neural network.

Clause 36. A processor-implemented method of soft-tissue-labeling a computed tomography (CT) image, the method comprising:
providing an input CT image as input to the neural network of clause 34; and
receiving, from the neural network, a soft-tissue-labeling of the input CT image.

Clause 37. A system for labeling soft-tissue in a computed tomography (CT) image, the system comprising:
at least one processor; and
a memory coupled to the at least one processor, the memory storing instructions that, when executed by the at least one processor, cause the at least one processor to:
provide an input CT image as input to the neural network of clause 34; and
receive, from the neural network, a soft-tissue-labeling of the input CT image.

Clause 38. The processor-implemented method of clause 26, wherein the soft-tissue is cartilage.

What is claimed is:

1. A processor-implemented method of training a neural network for soft-tissue labeling comprising:
creating a training set of soft-tissue-labeled computed tomography (CT) images comprising:
collecting, from at least one database, pairs of images for each of a plurality of individuals, each pair comprising both a CT image of an individual's anatomy and a magnetic resonance (MR) image of the individual's anatomy; and
for each of the pairs:
generating a 2D transformation between bone in the MR image and bone in the CT image;
labeling soft-tissue in the CT image based on an application of the 2D transformation to labeled soft-tissue in the MR image, thereby to create a soft-tissue-labeled CT image; and
augmenting the training set with the soft-tissue-labeled CT image; and
training the neural network using the training set to label soft-tissue in CT images.

2. The processor-implemented method of claim 1, wherein labeling soft-tissue in the CT image based on an application of the 2D transformation to labeled soft-tissue in the MR image comprises:
applying the 2D transformation to the soft-tissue in the MR image to label contents of the CT image as soft-tissue thereby to create the soft-tissue-labeled CT image.

3. The processor-implemented method of claim 1, wherein generating a 2D transformation between the bone in the MR image and the bone in the CT image comprises:
applying a global registration process to determine an initial alignment transformation between the bone in the MR image and the bone in the CT image; and
applying an iterative closest point (ICP) process to the initial alignment transformation thereby to generate the 2D transformation.

4. A non-transitory processor-readable medium embodying processor-readable program code executable by at least one processor to carry out the processor-implemented method of claim 1.

5. A neural network trained according to the processor-implemented method of claim 1.

6. A processor-implemented method of soft-tissue-labeling a computed tomography (CT) image, the method comprising:
providing an input CT image as input to the neural network of claim 5; and
receiving, from the neural network, a soft-tissue-labeling of the input CT image.

7. A system for labeling soft-tissue in a computed tomography (CT) image, the system comprising:
at least one processor; and
a memory coupled to the at least one processor, the memory storing instructions that, when executed by the at least one processor, cause the at least one processor to:
provide an input CT image as input to the neural network of claim 5; and
receive, from the neural network, a soft-tissue-labeling of the input CT image.

8. The processor-implemented method of claim 1, wherein the soft-tissue is cartilage.

9. A processor-implemented method of soft-tissue labeling an input CT image of an individual's anatomy comprising:
generating, by a neural network based on the input CT image, a respective output synthetic magnetic resonance (MR) image of the individual's anatomy, wherein the neural network is trained using a generative adversarial approach, using at least CT training images and MR training images, to receive CT images of individuals' anatomy and generate respective output synthetic MR images of the individuals' anatomy;
receiving at least soft-tissue labeling corresponding to the respective output synthetic MR image; and
applying at least the soft-tissue labeling from the respective output synthetic MR image to the input CT image thereby to soft-tissue label the input CT image.

10. The processor-implemented method of claim 9, wherein receiving at least soft-tissue labeling corresponding to the output synthetic MR image comprises:
providing the output synthetic MR image to an automatic image segmentation system; and
receiving at least the soft-tissue labeling corresponding to the output synthetic MR image from the automatic image segmentation system.

11. The processor-implemented method of claim 10, wherein applying at least the soft-tissue labeling from the output synthetic MR image to the input CT image thereby to soft-tissue label the input CT image comprises:
resampling a segmentation volume of the output synthetic MR image using the input CT image as a reference.

12. The processor-implemented method of claim 9, further comprising:
configuring the neural network as a first generative adversarial network (GAN) interconnected with a second GAN, wherein the first GAN is trained to generate a synthetic output MR image from a first GAN input, and the second GAN is trained to generate a synthetic CT image from a second GAN input; and
training the neural network by, for each of a plurality of cycles:
providing, as a first GAN input, a synthetic CT image generated by the second GAN thereby to generate, as a first GAN output, a corresponding synthetic MR image;
providing, as a second GAN input, a synthetic MR image generated by the first GAN thereby to generate, as a second GAN output, a corresponding synthetic CT image;
determining a first measure of difference between the second GAN output and the first GAN input;
determining a second measure of difference between the first GAN output and the second GAN input; and
modifying the first GAN and the second GAN based at least on the first measure of difference and the second measure of difference, wherein the modifying is conducted to reduce, over successive cycles, magnitudes of the first measure of difference and the second measure of difference.

13. The processor-implemented method of claim 12, further comprising:
during training of the neural network:
receiving bone labeling for the synthetic CT image generated by the second GAN; and
using the bone labeling as regularization during the modifying.

14. The processor-implemented method of claim 9, wherein the individual's anatomy is a knee joint and the soft-tissue labeling includes labeling for femoral cartilage and labeling for tibial cartilage.

15. A non-transitory processor-readable medium embodying processor-readable program code executable by at least one processor to carry out the processor-implemented method of claim 9.

16. A neural network trained according to the processor-implemented method of claim 9.

17. The neural network of claim 16, wherein the neural network is a U-net neural network.

18. A processor-implemented method of soft-tissue-labeling a computed tomography (CT) image, the method comprising:
providing an input CT image as input to the neural network of claim 16; and
receiving, from the neural network, a soft-tissue-labeling of the input CT image.

19. A system for labeling soft-tissue in a computed tomography (CT) image, the system comprising:
at least one processor; and
a memory coupled to the at least one processor, the memory storing instructions that, when executed by the at least one processor, cause the at least one processor to:
provide an input CT image as input to the neural network of claim 16; and
receive, from the neural network, a soft-tissue-labeling of the input CT image.

20. The processor-implemented method of claim 9, wherein the soft-tissue is cartilage.
